(19) United States
(12) Patent Application Publication
(10) Pub. No.: US 2023/0027100 A1
(43) Pub. Date: Jan. 26, 2023

(54) METHOD AND SYSTEM FOR DIGITAL ATTESTATION

(71) Applicant: GEROMICS LTD, Cambridge (GB)

(72) Inventors: Daniel Murray Bolser, Cambridge (GB); Carlos Massiah, Cambridge (GB)

(21) Appl. No.: 17/788,354

(22) PCT Filed: Dec. 23, 2020
(86) PCT No.: PCT/GB2020/053351

§ 371 Date: Jun. 23, 2022

(30) Foreign Application Priority Data
Dec. 24, 2019 (GB) 1919284.8

(51) Int. Cl. G06F 21/62; G06F 21/64; G06F 21/60

(52) U.S. Cl. CPC G06F 21/6245; G06F 21/645; G06F 21/602

(57) ABSTRACT

Broadly speaking, embodiments of the present techniques provide methods and systems to enable a user to securely share user information with a third party. The user information is based on a user data item, but the user data item itself is kept secret and not shared with the third party. The present techniques generate a digital attestation or verifiable credential containing the user information to Receiving an attestation request for a fact be shared.

The present techniques generally relate to systems and methods for securely and privately sharing user information with a third party. The user information being shared may be genome data or other personal data.

Individuals may wish to share some information with a third party, without revealing too much information or sharing too much personal information, i.e. while preserving privacy.

Background information can be found in the following prior art documents: EP3340149 which relates to methods and systems for validating an interaction between a user and a service provider by transferring user credential information about a user to the requester; US2019/121813 which relates to attesting to a fact providing provided by the user based on evidentiary support provided by the user; WO2019/217937 which relates to an identity verification system that authenticates personal information; and US2012/0167235 which relates to verifying one or more facts from personal data associated with a user. However, none of these documents solve the above-mentioned problem that a user may wish to or need to share some personal information with a third party while preserving privacy. For example, in many countries, a passport, national identity card or driver's license may be requested by a third party in order to confirm a user's identity, because these are issued by a trusted governmental agency. However, these types of ID typically contain lots of personal information which is then unnecessarily revealed to the third party (or even stored by the third party). The above-mentioned prior art documents do not enable a user to maintain their privacy.

Thus, the present applicant has identified the need for a system that allows users to securely and privately share user information while preserving privacy.

In a first approach of the present techniques, there is provided a method for securely sharing user information with a third party, the method comprising: receiving an attestation request from a third party for at least one fact about a user; obtaining at least one user data item associated with the user; determining at least one fact from the user data item; generating an attestation of the at least one fact; and transmitting the generated attestation. The attestation request may be received from the third party directly or may be received via the user.

In other words, the present techniques enable a user to share certain pieces of user information or certain facts, without revealing all of the data containing the user information or the data from which the facts are derived. For example, a user may wish to reveal certain facts that derive from their DNA sequence data or genome data, without revealing the full DNA sequence or genome data itself. This is because the DNA sequence or genome data is highly sensitive and private, and a user may wish to keep this secret, but may wish to obtain information or services based on facts derived from the data.

For example, a whole genome sequence may identify details of a user's ability to metabolise opioids and the user's tendency towards addiction. In combination, these two facts derived from the genome sequence could enable the user to safely access prescriptions for medical drugs that a doctor or pharmacist would otherwise be hesitant to prescribe. The user would not want to provide their full genome sequence to a pharmacist in order to obtain the prescription, and a pharmacist may not trust the data provided by the user anyway. The present techniques enable an attestation module or authority to issue certificates, i.e. attestations, about these facts—a pharmacist would trust the attestation as it has been generated by a trusted authority, and the user would be able to maintain the secrecy and privacy of some aspects of their data while revealing enough information to access the product or service they require.

The attestation may further comprise one or more of: context information, an issuance date and time stamp, an expiry date and time stamp, and a cryptographic proof.

The identified user data item may comprise any one of: genome data, health data, lifestyle data, location data, personal data, and biometric data.

Each user data item may comprise a plurality of parts. The step of determining at least one fact from the user data item may comprise: extracting at least one part from the user data item; and using the extracted at least one part as the at least one fact. Alternatively, the step of determining at least one fact from the user data item may comprise: deriving, from at least one part of the user data item, the at least one fact. Determining the at least one fact from the user data item may comprise using an algorithm (e.g. an AI algorithm trained to identify facts from user data items) or may be comprise using heuristic techniques.

The step of obtaining the at least one user data item may comprise requesting the user to provide the at least one user data item. Alternatively, the step of obtaining the at least one user data item may comprise requesting the user data item from a secure storage.

In some or all cases, the method comprises: receiving a Distributed IDentity (DID) from the user; and using the DID to obtain information identifying the user for generating the attestation.

In a second approach of the present techniques, there is provided a system for securely sharing user information with a third party, the system comprising: an attestation module, and a data access platform comprising storage for storing a plurality of user data items.

The attestation module may transmit, to the data access platform, an attestation request for at least one fact about a user. The data access platform may: receive the attestation request; obtain at least one user data item associated with the user from the storage; determine at least one fact from the at least one user data item; generate an attestation of the at least one fact; and transmit the generated attestation to the attestation module.

The attestation module may use the received generated attestation to determine whether the at least one fact about the user is correct.

In a related approach of the present techniques, there is provided a non-transitory data carrier carrying processor control code to implement any of the methods, processes and techniques described herein.

As will be appreciated by one skilled in the art, the present techniques may be embodied as a system, method or computer program product. Accordingly, present techniques may take the form of an entirely hardware embodiment, an entirely software embodiment, or an embodiment combining software and hardware aspects.

Furthermore, the present techniques may take the form of a computer program product embodied in a computer readable medium having computer readable program code embodied thereon. The computer readable medium may be a computer readable signal medium or a computer readable storage medium. A computer readable medium may be, for example, but is not limited to, an electronic, magnetic, optical, electromagnetic, infrared, or semiconductor system, apparatus, or device, or any suitable combination of the foregoing.

Computer program code for carrying out operations of the present techniques may be written in any combination of one or more programming languages, including object oriented programming languages and conventional procedural programming languages. Code components may be embodied as procedures, methods or the like, and may comprise sub-components which may take the form of instructions or sequences of instructions at any of the levels of abstraction, from the direct machine instructions of a native instruction set to high-level compiled or interpreted language constructs.

Embodiments of the present techniques also provide a non-transitory data carrier carrying code which, when implemented on a processor, causes the processor to carry out any of the methods described herein.

The techniques further provide processor control code to implement the above-described methods, for example on a general purpose computer system or on a digital signal processor (DSP). The techniques also provide a carrier carrying processor control code to, when running, implement any of the above methods, in particular on a non-transitory data carrier. The code may be provided on a carrier such as a disk, a microprocessor, CD- or DVD-ROM, programmed memory such as non-volatile memory (e.g. Flash) or read-only memory (firmware), or on a data carrier such as an optical or electrical signal carrier. Code (and/or data) to implement embodiments of the techniques described herein may comprise source, object or executable code in a conventional programming language (interpreted or compiled) such as C, or assembly code, code for setting up or controlling an ASIC (Application Specific Integrated Circuit) or FPGA (Field Programmable Gate Array), or code for a hardware description language such as Verilog® or VHDL (Very high speed integrated circuit Hardware Description Language). As the skilled person will appreciate, such code and/or data may be distributed between a plurality of coupled components in communication with one another. The techniques may comprise a controller which includes a microprocessor, working memory and program memory coupled to one or more of the components of the system.

It will also be clear to one of skill in the art that all or part of a logical method according to embodiments of the present techniques may suitably be embodied in a logic apparatus comprising logic elements to perform the steps of the above-described methods, and that such logic elements may comprise components such as logic gates in, for example a programmable logic array or application-specific integrated circuit. Such a logic arrangement may further be embodied in enabling elements for temporarily or permanently establishing logic structures in such an array or circuit using, for example, a virtual hardware descriptor language, which may be stored and transmitted using fixed or transmittable carrier media.

In an embodiment, the present techniques may be implemented using multiple processors or control circuits. The present techniques may be adapted to run on, or integrated into, the operating system of an apparatus.

In an embodiment, the present techniques may be realised in the form of a data carrier having functional data thereon, said functional data comprising functional computer data structures to, when loaded into a computer system or network and operated upon thereby, enable said computer system to perform all the steps of the above-described method.

Implementations of the present techniques will now be described, by way of example only, with reference to the accompanying drawings, in which:

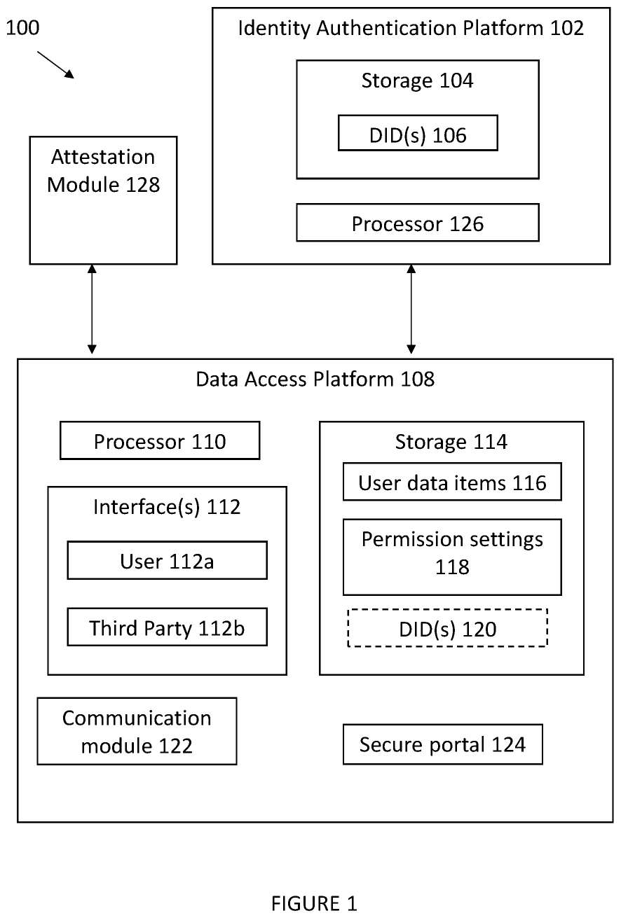

FIG. 1 shows a block diagram of a system for securely sharing user data items;

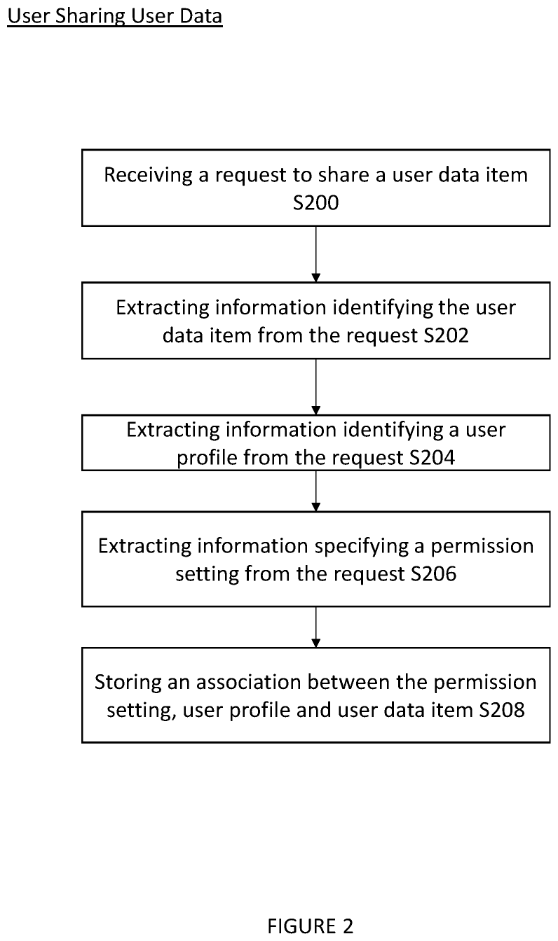

FIG. 2 shows a flowchart of example steps to enable a user to securely share their user data;

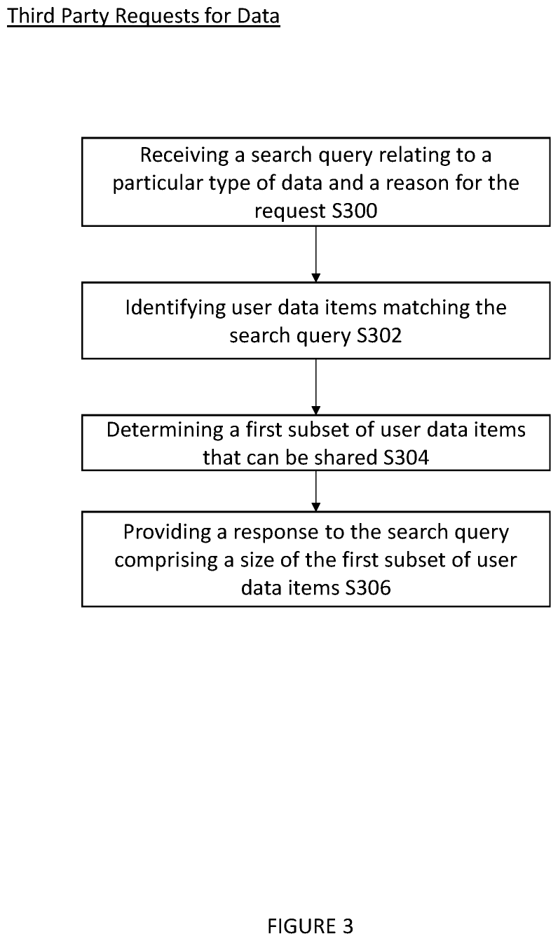

FIG. 3 shows a flowchart of example steps to enable a third party to access user data items;

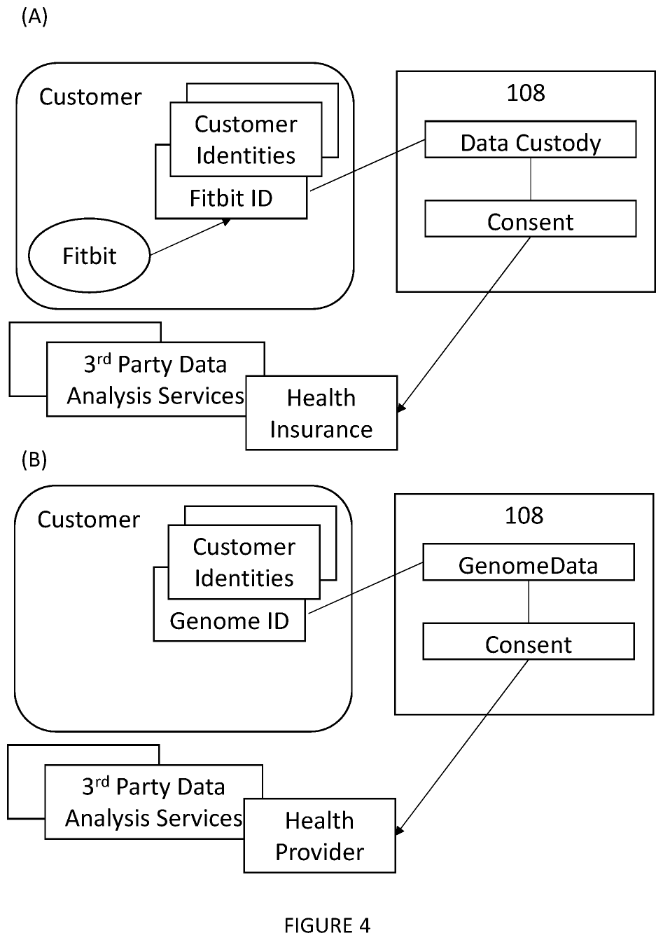

FIG. 4A and FIG. 4B show examples of how a user can control access to their user data items by different third parties using different profiles;

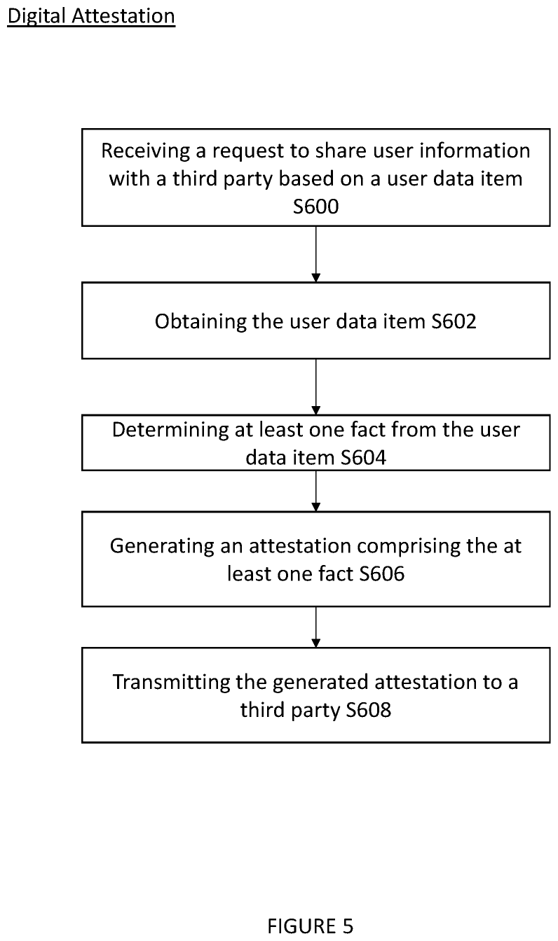

FIG. 5 shows a flowchart of example steps to generate a digital attestation; and

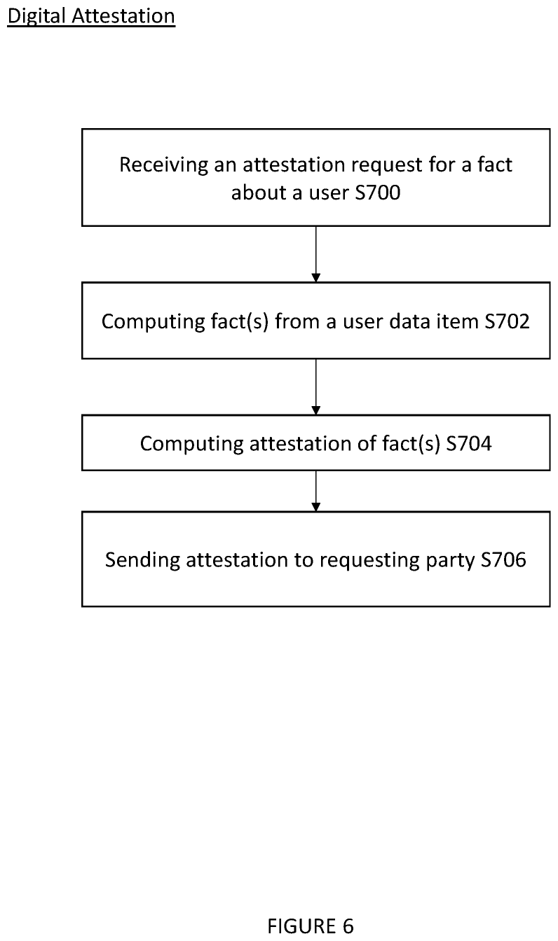

FIG. 6 shows a flowchart of example steps to generate a digital attestation requested by a third party.

Broadly speaking, embodiments of the present techniques provide methods and systems to enable a user to securely share user information with a third party. The user information is based on a user data item, but the user data item itself is kept secret and not shared with the third party. The present techniques generate a digital attestation or verifiable credential containing the user information to be shared.

The digital attestation generation may be used in conjunction with a system that enables a user to share their data with selected third parties in a way that ensures the user retains at least some control over their data at all times. Thus, techniques described herein may enable a user to benefit from the results of sharing their data (e.g. access to health related products or services, kudos or a reward), without revealing details of or losing control of their data and without the possibility of unauthorised use of their data. A user may add user data items into a storage and may specify for each user data item a permission setting that specifies whether or not the user data item may be shared, who it can be shared with or for what purpose it can be shared. A third party who is granted access to a user data item is able to access the user data item via a secure portal, but is not permitted to remove, download or copy the data item from the storage itself. Thus, unauthorised use or sharing of user data is prevented.

FIG. 1 shows a block diagram of a system 100 for securely sharing user data items with third parties. The system may comprise a data access platform 108. The data access platform 108 enables users to store user data items into a secure storage, and enables third parties to submit queries requesting access to particular user data items or to submit attestation requests regarding facts about a user. The data access platform 108 may be accessed by users and third parties in any suitable way, e.g. via a software application (‘app’) or via a web browser.

The data access platform 108 may comprise storage 114 for storing a plurality of user data items 116 and at least one permission setting 118 associated with each user data item. In some cases, storage 114 may comprise a DID document associated with a user profile/account, or may comprise a pointer to each DID document stored elsewhere in system 100. The storage 114 may comprise a volatile memory, such as random access memory (RAM) for use as temporary memory, and/or non-volatile memory such as Flash, read only memory (ROM), or electrically erasable programmable ROM (EEPROM), for storing data, programs, instructions, etc. The storage 114 may comprise one or more databases.

The storage 114 may store user data items in a secure, encrypted format. In some cases, user data items may be custodied with multiple partners. That is, partners may store user data items in an encrypted form, but a single partner does not hold a complete set of data or a complete user data item, such that if one data store is breached, data remains secure. Thus, user data items may be split, encrypted and stored in multiple data stores managed by data custodians.

The data access platform 108 may comprise a plurality of interfaces 112 that enable the data access platform to receive inputs (e.g. requests from a user or third party) and to generate outputs (e.g. responses to the requests, or feedback to the user on how their data is being used). One of the plurality of interfaces 112 may be a user interface 112a for receiving, from a user, a request to share a user data item. The user interface 112a may be used to send information back to the user, e.g. to show them how their user is being used or who has accessed their data, to prompt the user to share their user data, and to provide the user with a reward when their user data has been shared with/accessed by a third party. Another of the plurality of interfaces 112 may be a third party user interface 112b for receiving, from a third party, a search query relating to a particular type of user data and a reason for wanting the user data. The third party user interface 112b may be used to send information back to the third party, e.g. to provide them with a response to their query, to provide them with the option to obtain access to user data items, and to provide them with access to the user data items.

The data access platform 108 may comprise at least one processor 110 or processing circuitry for carrying out any of the methods described herein. The processor 110 controls various processing operations performed by the data access platform 108, such as communicating with other components of the platform 108, communicating with other components within system 100, and processing requests received from users and third parties. The processor may comprise one or more of: a server, a microprocessor, a microcontroller, and an integrated circuit.

The data access platform 108 may comprise a communication module 122 to enable the platform 108 to communicate with other components of the system 100, or to communicate with companies in order to request and/or receive user data from the companies for storage in storage 114.

The data access platform 108 may comprise a secure portal 124, to enable a third party to access a subset of the user data items 116 stored in storage 114.

The system 100 may further comprise an identity authentication platform 102 comprising: storage 104 storing a plurality of distributed identity documents (DIDs) 106, wherein at least one DID 106 is associated with each user of the system 100.

The identity authentication platform 102 may further comprise at least one processor or processing circuitry 126. The processor 126 controls various processing operations performed by the identity authentication platform 102, such as communicating with other components of the platform 102, communicating with other components within system 100, and processing requests received from users to create user profiles, create DIDs, move or delete DIDs, etc. The processor 126 may comprise one or more of: a server, a microprocessor, a microcontroller, and an integrated circuit.

The data access platform 108 provides secure data storage to users, with full rights to and control over their data. The data access platform 108 may allow them to profit from their data assets. Each user may use a blockchain-based DID to interact with the data access platform 108, but this is not required and other types of DIDs may be used. Organisations wishing to use user data for research purposes may be able to use the data access platform 108 to find users and user data items (depending on the permission settings of the user data items) for their research. Analysis service providers may be able to market their products and services to users of the platform 108. For example, if a user has stored genome information or health information as user data items in storage 114, analysis service providers may be able to offer the user the chance to have their data analysed.

Users may benefit from the system 100 in a number of ways. For example, the system may empower users to unleash the potential of their genomic, clinical, medical, and/or lifestyle data. The system may enable users to obtain personalised interpretation and analysis of their data from data service providers. The system may enable users to monetise their data (e.g. private genomic data) to healthcare companies, while retaining ownership of the data. In one example, users who have their full genome sequence data may be able to open an account to access system 100 and store their data securely within system 100. Users of the platform 108 may be able to buy services that perform analysis on their data, such as cancer screening or ancestry discovery, while retaining full ownership of their data. Users have the option to consent to their data being used in research by other companies, and of earning rewards (such as kudos, monetary or non-monetary rewards) when their data is used.

Third parties, such as research organisations, may benefit from the system 100 in a number of ways. For example, the system may enable researchers to connect with specific users who—based on their user data—may be identified as being suitable to participate in a clinical trial. The system may remove the barrier of separate siloed genome or health data, as it offers the potential for researchers to gain access to many items of data.

Analysis service providers (e.g. genome analysis or lifestyle analysis companies) may benefit from the system 100 in a number of ways. For example, such companies may be able to provide tailored services to a user based on the user's genomic or clinical data. The system may enable streamlining of business processes, and remove any risks associated with data custody, as the user data remains in the system 100. The companies gain access to a vast array of users who may already have access to their full genome, and they can therefore provide the users with specific analysis services.

The system 100 may comprise an attestation module 128. The attestation module 128 may transmit an attestation request for at least one fact about a user to the data access platform 108. The attestation module may be implemented in hardware, software (e.g. a program) or a combination of both hardware and software.

In embodiments, the attestation module 128 may be used to validate attestations produced/generated by the data access platform 108. For example, a third party system or device (e.g. a payment device in a pharmacy) may request attestation of one or more user facts before the third party can provide a service to the user (e.g. sell them particular drugs/medicines). Thus, the third party may submit a request to the data access platform 108 via attestation module 128. The third party may own the attestation module This is also explained with reference to FIG. 6. In embodiments, the attestation module 128 may generate the attestations itself, and therefore, the attestation module 128 may obtain the identified user data item from the storage 114 of the data access platform 108. This is also explained with reference to FIG. 5. The user may need to change the permissions settings associated with the identified user data item in order for the attestation module 128 to obtain the data item.

FIG. 2 shows a flowchart of example steps to enable a user to securely share their user data with third parties. The method may comprise receiving, from a user, a request to share a user data item (step S200). The request may be received via the user interface 112a of the data access platform 108. The received request may comprise: information identifying a user data item to be shared, information identifying a user profile for sharing the user data item, and information specifying a permission setting for the user data item to be shared. At steps S202 to S206, the method may comprise extracting these individual pieces of information from the request. The method may comprise storing, in storage 114, an association between the permission setting, the identified user profile and the user data item.

As mentioned above, the present techniques provide a user with the ability to securely share their data with third parties in a controlled manner. The user may have a number of user data items (such as health data, genomic data, and biometric data), and the user may wish to share the user data items with different third parties. For example, the user may wish to share their health data with a medical insurance company, but does not want to share this data with any other company. The system 100 enables users to share their data using a range of consent options or permission settings. The present techniques enable a user to specify a permission setting for each user data item that is stored in the storage 114. For example, some users may not be happy for their data to be shared, while some users may be happy to share their data with any University research organisation. Thus, the permission settings enable a user to have granular control over their data. The permission setting may specify that a particular user data item can be accessed by any third party (i.e. available to all), or cannot be accessed by any third party (i.e. available to none), or may specify access controls between these two extremes.

A distributed identity (also known as a decentralised identity) gives users the ability to control their login information used to access platform 108. An advantage of using DIDs is that businesses/companies do not need to store personal data to identify users or enable a user to recover their account when they have forgotten their login details (e.g. by storing phone numbers, the user's mother's maiden name, etc.) Furthermore, DIDs enable users to create multiple user accounts or profiles each linked to or associated with a single unique identity This provides a user with an additional degree of control over their personal and private data.

As explained in more detail below, once a user has created a single unique identity that gives them access to the storage in which they can store their user data items, the user may be able to create multiple profiles or accounts that are associated with that single unique identity. For example, the user may wish to create a different profile for each service provider or third party that they want to share some of their data with. For example, the user may create one profile for medical insurance providers, another profile for research organisations, another profile for healthcare professionals, and so on. The profiles may enable a user to apply general/global permission settings to their user data based on the type of third party. Each profile may be associated with a distributed identity (DID) document. The DID document corresponding to a profile may comprise information on how the system is to interact with the profile and a method for the user to prove they own the profile. As stated above, when a user requests to share a user data item, they may provide information identifying a user profile for sharing the user data item. The information identifying a user profile for sharing the user data item may be one of: a distributed identity, distributed identity (DID) document, or a pointer to a distributed identity (DID) document. Thus, when the user makes a request to share a user data item, the user provides information that authenticates them and verifies that they are allowed to share the user data item.

The storage 114 stores user data items 116. However, the request to share a user data item 116 may be received before that user data item has been stored in the storage. Thus, the method may comprise determining whether the identified user data item (specified in the request) is available in the storage and available to the user for sharing.

If the user data item is identified in storage 114 and is available to the user for sharing, the method may comprise associating the identified user data item with the stored permission setting. This may be the case if the user has already obtained the data item and saved it in the storage 114. The user may have obtained (e.g. by purchasing) the data item from a third party, and saved the data into the storage 114. In some cases, the user may have purchased the data item from a third party, and the third party may have saved the data into storage 114 on behalf of the user. For example, a third party company may provide data as a service (e.g. genomic analysis), and may have a relationship with the storage provider that allows them to put the data they generate for users/customers into the storage, so that the user can access their own data directly from the storage 114.

Alternatively, the user data item may be available in storage 114 but may not be available to the user for sharing. This may be the case if, for example, a third party company has stored data they generated for users/customers in the storage 114, but the user has not yet taken the necessary steps to obtain or claim that data. Thus, the method may comprise: providing the user with an option to obtain the identified user data item; and associating, when the user obtains the identified user data item, the identified user data item with the stored permission setting.

Alternatively, if the user data item is not available in the storage 114 (e.g. because the request to share has been made before the data item has been added to the storage), the method may comprise requesting the identified user data item from the user.

The user may be able to specify any permission setting for each user data item associated with/belonging to the user in the storage. For example, the information (in the request) specifying a permission setting for the user data item may comprise any one or more of: information restricting access to the user data item to a specific third party, information specifying the user data item can be used by a predefined set of third parties, information specifying that the user data item can be used for private research, information specifying that the user data item can be used for public research, and information specifying that the user data item can be used for private and public research. It will be understood that this is a non-exhaustive and non-limiting list of example permission settings, and that other more specific permission settings may be used.

The user data item to be shared may comprise any one of: genome data, health data, lifestyle data, location data, personal data, and biometric data. It will be understood that this is a non-exhaustive and non-limiting list of example types of user data.

As mentioned above, third parties may be able to submit queries, via the third party interface 112b, to determine if data they need for research or analytics purposes is stored in the storage. However, the third parties cannot see or access any particular user data item unless the user associated with the user data item has expressly provided them with permission to do so. In response to a query received from a third party, it may desirable to provide the third party with information indicating whether any data matching their query exists in the storage 114. However, even if data matching the query exists in the storage 114, the data may not be available to the third party because of the permission settings set by each user. Thus, it may be advantageous to separate the data stored in the storage into two groups: searchable data and non-searchable data. The searchable data includes the user data items that users have explicitly stated may be searched by a third party. The non-searchable data includes the user data items that users have stated may not be searched by a third party. Thus, when a third party queries the system, they are only able to do so with respect to the user data items that are marked/categorised as being searchable. Thus, the method may comprise: adding the user data item to be shared to a database of searchable data, based on the permission setting.

As the system enables a user to have full control over their data, the method may further comprise: receiving, from the user, a request to revoke access by third parties to a previously shared user data item. In response to this request, the method may comprise removing the previously shared user data item from a database of searchable data. Alternatively, the method may comprise deleting, responsive to the request, the previously shared user data item from the storage.

FIG. 4A and FIG. 4B show examples of how a user can control access to their user data items by different third parties using different profiles. In FIG. 4A, a user may store data collected by a smartwatch, sports watch or fitness tracking device (such as a Fitbit), in the platform 108. The user may have a number of user profiles and associated DIDs. Here, the user has a user profile and DID specifically for their Fitbit data. When a third party such as an insurance company wants access to the user's fitness or health data (as collected by the fitness tracking device), the request may be made to the platform 108. The user may request to share user data items corresponding to their fitness/health, or corresponding to their Fitbit user profile, and may specify a permission setting that allows the insurance company to access these specific user data items. Once the consent has been provided, the third party may be able to view/access the user data items via a secure portal.

In FIG. 4B, the same user is shown as have a user profile and DID for their genome data. A third party, such as a healthcare provider, may want access to the user's genome data. For example, researchers may want to survey users who have a specific genotype, but these users may only be identified by first accessing and analysing users' genome data. Again, the user may specify a permission setting using their genome user profile to enable the third party to access the user data items that are associated with the genome user profile. Once the consent has been provided, the third party may be able to view/access the genome data via a secure portal.

Thus far, methods for how a user may share user data items have been described. However, the present techniques also provide methods for enabling a third party to obtain access to user data items that a user has permitted to be shared.

There may be two ways to use the system 100 to provide business and researchers with access to datasets. One way comprises providing the third parties with access to platform 108 so that they can query whether any data exists that matches the criteria they need for their research. This is explained below with reference to FIG. 3. Another approach would be to approach companies who are looking for data with relevant datasets (taking into account the users' permission settings), and offer the datasets to the companies as a package. In either case, the user data items in the datasets are anonymised.

Turning to FIG. 3, this shows a flowchart of example steps to enable a third party to access user data items.

Third parties may be able to buy access to user data items via the data access platform 108, e.g. via a secure web portal 124, and to view the data through e.g. a REST API.

Third parties may be able to submit surveys, buy data and manage keys through the secure portal 124. If a third party wishes to purchase user data items, the third party is provided with an access token that enables them to view the user data items via the REST API.

The method may comprise receiving, from a third party (via the third party interface 112b), a search query relating to a particular type of user data and a reason for wanting the user data (step S300). As mentioned above, a user may specify that a specific data item can only be accessed for a particular use/purpose (e.g. private and/or public research) or by a particular type of organisation (e.g. Universities, research organisations, non-profit organisations, commercial companies, etc.) Thus, the third party may have to specify a reason why they want to access the user data items, so that only those user data items that are permitted to be shared for that reason are used to provide a response to the query.

The third party may wish to find data that relates to their research endeavours. For example, a skin cream manufacturer may be looking for phenotype data in people who have a specific gene related to a possible skin type. In another example, a study correlating specific genes to an increased incidence of a certain disease may need human genome data from anyone who has that disease.

Such queries are performed within platform 108. Thus, the method may comprise identifying, in storage 114, a plurality of user data items matching the search query (step S302). At step S304, the method may comprise determining, using permission settings associated with each user data item and the received reason, a first subset of user data items that can be shared with the third party.

A first response to the query may simply be a number equal to the number of user data items in the first subset (i.e. which match the received search query and have permissions settings corresponding to the received reason). This may help the third party to determine if they want to proceed further and request full access to the user data items. For example, if only a small number of user data items are identified, then the third party may not wish to obtain full access to the user data items, as the number may be too small to analyse and reach meaningful conclusions. Thus, at step S306 the method may comprise providing, to the third party, a response to the search query comprising a size of the first subset of user data items that match the received search query. In some cases, the response may comprise the size of the first subset of user data items, and some samples or example user data items that match the search query. User data items in the first subset may be added to a dataset, and access to the dataset that could be purchased by the third party through the data access platform 108.

The third party may be able to use user data items according to terms set by the owner of system 100. For example, the third party may be able to access data only for the duration that the third party needs access. That is, when the third party's research has ended, they will no longer be able to access the user data items. The third party may also be prevented from disclosing the user data items to any other party for any reason.

The method may comprise: receiving, from the third party, a request to access the first subset of user data items; and providing the third party with an access token to enable the third party to access the first subset of user data items through a secure portal 124. In some cases, the access token may enable the third party to view the first subset of user data items via the secure portal, but not to be able to download or copy the user data items. In this way, the user data items that are shared with a third party do not leave the storage and copies are not provided to the third party. Thus, the user retains full control of their user data at all times.

Third parties may be able to manage access control tokens for datasets that they have purchased via the data access platform 108. Access tokens may be added and removed. The tokens may enable the third parties to access specific data only, in a secure manner via secure portal 124.

It may be desirable to provide a user associated with each user data item accessed by a third party with feedback so that they know that a third party has accessed their data. For example, users may be provided with feedback on where or how their data is currently being used, such as the study their data is part of and the status of the study (e.g. completed or in progress). This provides users with information on how their data may be benefiting others. The method may comprise: identifying a user associated with each user data item of the first subset of user data items; and transmitting, to each identified user, a message indicating that a third party is accessing their user data item for the received reason.

Researchers/third parties may require follow-up information from users as part of their research. The system may enable users to be contacted. For example, researchers may submit their requests for additional information from the users whose user data items they have already accessed. The requests may be submitted via the third party interface 112b. The requests may be presented to each user, e.g. via the user interface 112a. The user may be able to submit a response or ignore the request via the user interface 112a. Thus, the data access platform 108 enables researchers to obtain further information from users while maintaining user privacy and ensuring the user has full control over what further information is shared.

In some cases, to encourage users to share more of their data, the method may comprise providing a reward, to each identified user associated with the first subset of user data items. For example, a user may be provided with a reward when they complete a survey submitted by a third party, or when access to their user data item(s) has been purchased by a third party. Monetary rewards may be provided to the users using cryptocurrency. This avoids the need for the system 100 to store bank details for the users. Such rewards may be made to the users immediately when a third party has paid for user data, so the owner of system 100 does not hold funds on behalf of the users.

The method may further comprise: determining a second subset of user data items that cannot be shared with the third party; identifying a user associated with each user data item of the second subset; and transmitting, to each identified user, a message indicating that a third party wants access to the user data item of the second subset associated with the user. Thus, the method enables users who have not already specified that the third party requesting the data can access their data with the opportunity to change the permission settings. This may be useful if, for example, a user has not provided any specific permission settings or has simply used default permission settings for their user data. By default, each user data item may be non-searchable and not shareable with any third party, to ensure the data remains private until the user specifies otherwise. However, if a user has not had time to change the permission settings, this method may prompt the user to do so in response to a third party request for particular data.

The size of the second subset of user data items, or any other information about the second subset may not be shared with the third party. However, if a user associated with a user data item in the second subset changes their permission settings to allow the third party to access the data item, the third party may be sent a message stating that another user data item has become available, and provide them with the option to obtain full access to the newly available user data item.

The method may further comprise: receiving, from the third party, a purchase request in relation to the first and/or second subset of user data items in the response.

The response to the search query may comprise a cost to access the subset of user data items. In this case, the method may further comprise: receiving, from the third party, a payment corresponding to the cost to access the subset of user data items; and providing the third party with an access token to enable the third party to access the first subset of user data items through secure portal 124.

The system 100 may further comprise a service market platform (not shown in FIG. 1). The service market platform may be provided within the data access platform 108, or may be communicatively coupled to platform 108. The service market platform may enable users to upload user data items, such as genotype data, and buy services that analyse the data. Example services include ancestry information, health analysis and cancer screening. Service providers may offer services through the platform 108 or the service market platform. The service providers may access the platform(s) via a web portal or interface.

FIG. 5 shows a flowchart of example steps to generate a digital attestation, which may be performed by the data access platform 108 and/or the attestation module 128. The method may enable a user to share certain pieces of user information or certain facts with a third party, without revealing all of the data containing the user information or the data from which the facts are derived.

For example, a user may need to provide proof of their identity to a third party. This is a common requirement when opening a new bank account or hiring a car, for instance. Currently a user may provide their passport, driver's license or national identity card in order to prove their identity, as these documents are typically issued by a trusted (governmental) agency, and usually contain a recent photograph of the owner of the document. The photograph is usually what the third party needs to check the identity of the user. However, the documents contain lots of other personal information about the user that the third party does not need to see and does not need to check the identity of the user, such as their date of birth, nationality, full home address, etc. As a result, currently, a user is unable to maintain their privacy. It would be helpful if a user could share certain information or certain facts with a third party, without revealing all their data.

In another example, a user may wish to reveal certain facts that derive from their full DNA sequence data or genome data, without revealing the DNA sequence or genome data itself. This is because the DNA sequence or genome data is highly sensitive and private, and a user may wish to keep this secret, but may wish to obtain information or services based on facts derived from the data.

For example, a whole genome sequence may identify details of a user's ability to metabolise opioids and the user's tendency towards addiction. In combination, these two facts derived from the genome sequence could enable the user to safely access prescriptions for medical drugs that a doctor or pharmacist would otherwise be hesitant to prescribe. The user would not want to provide their full genome sequence to a pharmacist in order to obtain the prescription, and a pharmacist may not trust the data provided by the user anyway.

The present techniques enable an attestation module or authority to issue certificates, i.e. attestations, about these facts—a pharmacist would trust the attestation as it has been generated by a trusted authority, and the user would be able to maintain the secrecy and privacy of some aspects of their data while revealing enough information to access the service they require.

Therefore, the present techniques are advantageous over current techniques which simply attest to the user data item rather than facts determined from the user data item. Furthermore, it avoids third parties receiving and storing copies of important documents, such as photocopies of user passports or national ID cards, which reduces the risk of user data becoming compromised if the third party's systems are subjected to a malicious attack. Another advantage is that the attestation is based on a fact determined from the user data item, rather than on evidence supplied by a user, which increases the trust in the attestation.

The method may comprise: receiving, from a user, a request to share user information with a third party, the request identifying a user data item as a source of the user information to be shared (step S600). The identified user data item may comprise any one of: genome data, health data, lifestyle data, location data, personal data, and biometric data.

The method may comprise obtaining the identified user data item (step S602). This step may comprise requesting the user to provide the identified user data item. Alternatively, the step of obtaining the identified user data item may comprise requesting the user data item from a secure storage.

The method may comprise determining at least one fact from the user data item (step S604). Each user data item may comprise a plurality of parts. The step of determining at least one fact from the user data item may comprise: extracting at least one part from the user data item; and using the extracted at least one part as the at least one fact. Alternatively, the step of determining at least one fact from the user data item may comprise: deriving, from at least one part of the user data item, the at least one fact.

The method may comprise generating an attestation comprising the at least one fact and information identifying the user (step S606), and transmitting the generated attestation to a third party (step S608). The attestation may further comprise one or more of: context information, an issuance date and time stamp, an expiry date and time stamp, and a cryptographic proof.

In some cases, the method may comprise: receiving a distributed identity (DID) from the user; and using the DID to obtain information identifying the user for generating the attestation.

FIG. 5 relates to the process which may be performed if a user wishes to send an attestation to a third party. FIG. 6 shows a flowchart of example steps to generate a digital attestation in response to a request by a third party. The process begins when the data access platform 108 receives a request from a third party to attest to a fact or facts about a user (step S700). The request may be received via the attestation module 128, or may be received directly from the third party. The processor 110 may extract at least one user data item 116 from storage 114 to compute one or more facts about the user from the stored data (step S702). The processor may compute an attestation for each computed fact (step S704). The processor 110 may send, using communication module 122, the computed attestation(s) to the third party which made the request (step S706) (either directly or via the attestation module 128). The third party or the attestation module 128 may then use the transmitted attestation to determine if the or each fact about a user is correct or not (i.e. is true or false).

The digital attestation idea may be used to share information with a wide variety of third parties. For example, a healthcare professional may perform a detailed diagnosis of a user's disability and generate a digital attestation (using a trusted authority) about the user's fitness to work. This digital attestation may be used to claim benefits at a benefits office without revealing the specific details of the user's disability. The detailed diagnosis may be saved in storage 114 as a user data item, and the information the user needs to share with the benefits office is based on the user data item. However, the user data item itself is not shared with the third party and thus, the user maintains control over their data.

In another example, a user may wish to share details of their skin type to a cosmetics company. The user's DNA may reveal details or information that can collectively be used by a genetics company to authoritatively assert the user's skin type. That attestation can be queried by skin care companies or cosmetics companies looking for people to market their products to. The companies may send products and services to the user based on the skin type, but the companies do not know any of the information used to determine the skin type or the DNA sequence of the user.

Those skilled in the art will appreciate that while the foregoing has described what is considered to be the best mode and where appropriate other modes of performing present techniques, the present techniques should not be limited to the specific configurations and methods disclosed in this description of the preferred embodiment. Those skilled in the art will recognise that present techniques have a broad range of applications, and that the embodiments may take a wide range of modifications without departing from any inventive concept as defined in the appended claims.

1. A method for securely sharing user information with a third party, the method comprising:
receiving, at a data access platform, an attestation request from a third party, via an attestation module, for at least one fact about a user, wherein the at least one fact is for use by the third party to determine the suitability of the user for a medical or cosmetic product provided by the third party;
obtaining, from storage, at least one user data item associated with the user, wherein the at least one user data item comprises genome data;
determining, at the data access platform, the at least one fact from the genome data, by deriving the at least one fact that indicates the suitability of the user for the medical or cosmetic product from at least one part of the genome data;
generating, at the data access platform, an attestation of the at least one fact; and
transmitting, from the data access platform, the generated attestation to the attestation module.

2. The method as claimed in claim, wherein the attestation further comprises one or more of: context information, an issuance date and time stamp, an expiry date and time stamp, and a cryptographic proof.

3. The method as claimed in claim 1, wherein the obtained user data item further comprises any one of: health data, lifestyle data, location data, personal data, and biometric data.

4. The method as claimed in claim 1, wherein each user data item comprises a plurality of parts, and wherein the step of determining at least one fact from the user data item comprises:
extracting at least one part from the user data item; and
using the extracted at least one part as the at least one fact.

5. The method as claimed in claim 1, wherein each user data item comprises a plurality of parts, and wherein the step of determining at least one fact from the user data item comprises:
deriving, from at least one part of the user data item, the at least one fact.

6. The method as claimed in claim 1, wherein the step of obtaining the at least one user data item comprises requesting the user to provide the identified user data item.

7. The method as claimed in claim 1, wherein the step of obtaining the at least one user data item comprises requesting the user data item from a secure storage.

8. A non-transitory data carrier carrying code which, when implemented on a processor, causes the processor to carry out the method of claim 1.

9. A system for securely sharing user information with a third party, the system comprising:
an attestation module for transmitting, to the data access platform, an attestation request for at least one fact about a user; and
a data access platform comprising storage for storing a plurality of user data items, wherein the data access platform:
receives the attestation request, wherein the at least one fact is for use by the third party to determine the suitability of the user for a medical or cosmetic product provided by the third party;
obtains at least one user data item associated with the user from the storage, wherein the at least one user data item comprises genome data;
determines the at least one fact from the genome data, by deriving the at least one fact that indicates the suitability of the user for the medical or cosmetic product from at least one part of the genome data;
generates an attestation of the at least one fact; and
transmits the generated attestation to the attestation module.

10. (canceled)

11. The system as claimed in claim 9, wherein the attestation module uses the received generated attestation to determine whether the at least one fact about the user is correct.
